SEQUENCE LISTING (30 sequences)

SEQ ID NO: 1 - protein, 735 aa, Bacillus anthracis
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 2 - protein, 258 aa, Bacillus anthracis
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAA

SEQ ID NO: 3 - protein, 229 aa, Bacillus anthracis
YPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQET

SEQ ID NO: 4 - protein, 108 aa, Bacillus anthracis
TARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKR

SEQ ID NO: 5 - protein, 140 aa, Bacillus anthracis
FHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 6 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 7 - protein, 29 aa, synthetic construct
MKKRKVLIPLMALSTILVSSTGNLEVIQA

SEQ ID NO: 8 - DNA, 2369 bp, synthetic construct
  source 1..2369: mol_type = other DNA
aatttcaatataatataaatttaattttatacaaaaaggagaacgtatatgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtcaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataaggtaattctaggtgatttttaaatta

SEQ ID NO: 9 - protein, 8 aa, synthetic construct
GSGESGSG

SEQ ID NO: 10 - protein, 9 aa, synthetic construct
GSGEESGSG

SEQ ID NO: 11 - protein, 9 aa, synthetic construct
GSGESGSGS

SEQ ID NO: 12 - protein, 10 aa, synthetic construct
GSGEESGSGS

SEQ ID NO: 13 - protein, 10 aa, synthetic construct
GGGGSGGGGS

SEQ ID NO: 14 - protein, 12 aa, synthetic construct
AHIVMVDAYKPT

SEQ ID NO: 15 - protein, 139 aa, synthetic construct
MSYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 16 - protein, 767 aa, Bacillus anthracis
MKYLLPTAAAGLLLLAAQPAMAMDIGINSDPMEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 17 - protein, 22 aa, synthetic construct
MKYLLPTAAAGLLLLAAQPAMA

SEQ ID NO: 18 - protein, 10 aa, synthetic construct
MDIGINSDPM

SEQ ID NO: 19 - protein, 1137 aa, synthetic construct
MKYLLPTAAAGLLLLAAQPAMAMDIGINSDPMEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIGSSTAQAQEWTPRSVTEIKSELVLVDNVFTYTVKYGDTLSTIAEAMGIDVHVLGDINHIANIDLIFPDTILTANYNQHGQATNLTVQAPASSPASVSHVPSSEPLPQASATSQPTVPMAPPATPSDVPTTPFASAKPDSSVTASSELTSSTNDVSTELSSESQKQPEVPQEAVPTPKAAETTEVEPKTDISEAPTSANRPVPNESASEEVSSAAPAQAPAEKEETSAPAAQKAVADTTSVATSNGLSYAPNHAYNPMNAGLQPQTAAFKEEVASAFGITSFSGYRPGDPGDHGKGLAIDFMVPENSALGDQVAQYAIDHMAERGISYVIWKQRFYAPFASIYGPAYTWNPMPDRGSITENHYDHVHVSFNA

SEQ ID NO: 20 - protein, 386 aa, synthetic construct
MGHHHHHHGSGENLYFQGSGQAQEWTPRSVTEIKSELVLVDNVFTYTVKYGDTLSTIAEAMGIDVHVLGDINHIANIDLIFPDTILTANYNQHGQATNLTVQAPASSPASVSHVPSSEPLPQASATSQPTVPMAPPATPSDVPTTPFASAKPDSSVTASSELTSSTNDVSTELSSESQKQPEVPQEAVPTPKAAETTEVEPKTDISEAPTSANRPVPNESASEEVSSAAPAQAPAEKEETSAPAAQKAVADTTSVATSNGLSYAPNHAYNPMNAGLQPQTAAFKEEVASAFGITSFSGYRPGDPGDHGKGLAIDFMVPENSALGDQVAQYAIDHMAERGISYVIWKQRFYAPFASIYGPAYTWNPMPDRGSITENHYDHVHVSFNA

SEQ ID NO: 21 - protein, 366 aa, Streptococcus pyogenes
QAQEWTPRSVTEIKSELVLVDNVFTYTVKYGDTLSTIAEAMGIDVHVLGDINHIANIDLIFPDTILTANYNQHGQATNLTVQAPASSPASVSHVPSSEPLPQASATSQPTVPMAPPATPSDVPTTPFASAKPDSSVTASSELTSSTNDVSTELSSESQKQPEVPQEAVPTPKAAETTEVEPKTDISEAPTSANRPVPNESASEEVSSAAPAQAPAEKEETSAPAAQKAVADTTSVATSNGLSYAPNHAYNPMNAGLQPQTAAFKEEVASAFGITSFSGYRPGDPGDHGKGLAIDFMVPENSALGDQVAQYAIDHMAERGISYVIWKQRFYAPFASIYGPAYTWNPMPDRGSITENHYDHVHVSFNA

SEQ ID NO: 22 - protein, 18 aa, Streptococcus pyogenes
AQEWTPRSVTEIKSELVL

SEQ ID NO: 23 - protein, 22 aa, Streptococcus pyogenes
ATPSDVPTTPFASAKPDSSVTA

SEQ ID NO: 24 - protein, 23 aa, Streptococcus pyogenes
EPKTDISEAPTSANRPVPNESAS

SEQ ID NO: 25 - protein, 324 aa, Streptococcus pyogenes
EWTPRSVTEIKSELVLVDNVFTYTVKYGDTLSTIAEAMGIDVHVLGDINHIANIDLIFPDTILTANYNQHGQATNLTVQAPASSPASVSHVPSSEPLPQASATSQPTVPMAPPATPSDVPTTPFASAKPDSSVTASSELTSSTNDVSTELSSESQKQPEVPQEAVPTPKAAETTEVEPKTDISEAPTSANRPVPNESASEEVSSAAPAQAPAEKEETSAPAAQKAVADTTSVATSNGLSYAPNHAYNPMNAGLQPQTAAFKEEVASAFGITSFSGYRPGDPGDHGKGLAIDFMVPENSALGDQVAQYAIDHMAERGISYVIWKQ

SEQ ID NO: 26 - protein, 80 aa, synthetic construct
SATHIKFSKRDEDGRELAGATMELRDSSGKTISTWISDGHVKDFYLYPGKYTFVETAAPDGYEVATPIEFTVNEDGQVTV

SEQ ID NO: 27 - protein, 114 aa, synthetic construct
MVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 28 - protein, 890 aa, synthetic construct
MKYLLPTAAAGLLLLAAQPAMAMDIGINSDPMEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIGGSGESGSGSMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 29 - protein, 855 aa, synthetic construct
MKYLLPTAAAGLLLLAAQPAMAMDIGINSDPMEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIGGSGESGSGSATHIKFSKRDEDGRELAGATMELRDSSGKTISTWISDGHVKDFYLYPGKYTFVETAAPDGYEVATPIEFTVNEDGQVTV

SEQ ID NO: 30 - protein, 4 aa, synthetic construct
STTA

(19) United States
(12) Patent Application Publication
(10) Pub. No.: US 2026/0053936 A1
(43) Pub. Date: Feb. 26, 2026

(54) IMMUNOGENIC PROTECTIVE ANTIGEN CONJUGATES

(71) Applicant: WICHITA STATE UNIVERSITY, Wichita, KS (US)

(72) Inventor: James BANN, Wichita, KS (US)

(21) Appl. No.: 19/305,208

(22) Filed: Aug. 20, 2025

Related U.S. Application Data
(60) Provisional application No. 63/685,096, filed on Aug. 20, 2024.

(51) Int. Cl. A61K 47/64; A61K 39/00; A61K 39/09

(52) U.S. Cl. CPC A61K 47/646; A61K 39/09; A61K 2039/6037

(57) ABSTRACT

The present disclosure is generally directed to an immunogenic protein conjugate comprising anthrax toxin protective antigen (PA) protein as an immune enhancer, a peptide linker, and an antigen derived from an infectious or pathogenic virus or microorganism. This protein conjugate can be used to induce an immune response in a subject, such as in a vaccine against the antigen of the protein conjugate. For example, the antigen can be Spy0469 for a vaccine against S. pyogenes.

### CROSS-REFERENCE TO RELATED APPLICATIONS

A present application claims the priority benefit of U.S. Provisional Patent Application Ser. No. 63/685,096, filed Aug. 20, 2024, entitled PROTECTIVE ANTIGEN CONJUGATE VACCINE, incorporated by reference in its entirety herein.

### SEQUENCE LISTING

The following application contains a sequence listing submitted electronically as a Standard ST.26 compliant XML file entitled “SequenceListing_62146-US.xml,” created on Aug. 20, 2025, as 36,611 bytes in size, the contents of which are incorporated herein.

### FIELD OF INVENTION

The present disclosure provides an immunogenic protein conjugate comprising anthrax toxin protective antigen (PA) protein as an immune enhancer, a linker, and an antigen conjugated to PA via the linker. This protein conjugate can be used in a vaccine or other immunogenic composition against the antigen of the protein conjugate. For example, the antigen can be Spy0469 for a vaccine against S. pyogenes.

### BACKGROUND OF INVENTION

Anthrax toxin is a member of the class of bacterial toxins termed AB toxins. AB toxins are composed of two moieties. The A moiety is the enzymic portion of the toxin that catalyzes the toxic effect upon a cytoplasmic target within a target cell. The B moiety binds to a cellular receptor and facilitates the translocation of the A moiety across the cell membrane into the cytoplasm of the cell.

The AB toxin secreted from Bacillus anthracis is comprised of the B moiety protective antigen (“PA”), and the A moieties edema factor (“EF”), and lethal factor (“LF”). The AB toxin disrupts host immune cells including dendritic cells. In particular, the binding component PA initiates cellular toxicity through high affinity binding to capillary morphogenesis protein 2 (CMG-2), a receptor expressed on dendritic cells, macrophages, and other antigen presenting cells. PA is a channel forming polypeptide that allows entry of EF and LF across membranes into the cell, a step that is critical for the pathogenesis of anthrax. PA is secreted as a four-domain, 83 kD protein that recognizes on the host cells the von-Willebrand factor A domain (“VWA”) of two integrin-like receptors: anthrax toxin receptor 1, (“ANTXR1”, formerly anthrax toxin receptor-tumor endothelial marker 8), and anthrax toxin receptor 2 (“ANTXR2”, formerly capillary morphogenesis protein 2 (“CMG-2”)). Binding of PA to the receptor results in the proteolytic cleavage of PA by a furin-like protease on the cell surface, releasing the first 167 amino acid residues of domain 1. Thus, the C-terminal 63 kDa fragment (“PA63”) remains bound to the cell and the N-terminal 20 kDa fragment (“PA20”) dissociates from PA63. This proteolytic cleavage and subsequent dissociation of PA20 confer at least two new properties on PA63: (1) the ability to oligomerize into a ring-shaped heptameric sodium dodecyl sulfate (“SDS”)-dissociable structure termed prepore and (2) the ability to bind EF and LF, which bind with a stoichiometry of three per heptameric prepore.

Binding of PA to the receptor also initiates receptor-mediated endocytosis into an endosomal compartment, which eventually becomes acidified. The low pH within the endosome induces a conformational change in the protective antigen that results in the formation of a membrane spanning channel, and this new conformation of the entire PA heptamer is termed the pore. The pore allows the transport of EF and LF into the cytosol. The exact pH required for pore formation is dependent upon interactions with the receptor—in vitro studies indicate that the pH is about 5 if the receptor is ANTXR2, and a slightly higher pH (about 6) if the receptor is ANTXR1. The receptor then dissociates from PA, allowing conformational changes to occur throughout the protein such that PA forms a membrane spanning pore.

Anthrax toxin PA is a major component of the anthrax vaccine (e.g., BioThrax, Emergent Biosolutions) and antitoxins (e.g., raxibacumab, Emergent Biosolutions).

Dendritic cells are the most potent antigen presenting cells and optimally induce activation and clonal expansion of naïve and memory T cells (CD4+ and CD8+) and direct the differentiation of their effector functions.

### SUMMARY OF INVENTION

The present disclosure is directed to a protein conjugate comprising anthrax toxin component protective antigen (PA), a linker, and an antigen. The high affinity binding of PA to CMG-2 expressed on dendritic cells is leveraged using PA as a targeting moiety or enhancer or antigen delivery vehicle or adjuvant to construct hybrid vaccines that are designed to optimize antigen presentation and T cell activation. For example, PA conjugates are used herein to deliver Spy0469, an antigenic surface protein of S. pyogenes that induces protective immunity in mouse models and is a potential vaccine against group A Streptococcus (GAS) infection. The high affinity binding of PA to CMG-2 (˜300 pM) has been shown to selectively target dendritic cells with an antigen from S. pyogenes known to induce an immune response in human cells. This is a protein conjugate vaccine against group A Streptococcus, which has not been previously developed. The exemplified antigen, Spy0469, can also be replaced with another antigen to generate other protein conjugate vaccines, such as pp65 (antigen from human cytomegalovirus), PsaA (antigen from Streptococcus pneumoniae), SpyTag003-OspC (antigen from Borrelia burgdorferi; Lyme Disease), NDV HN-SpyTag (antigen from Newcastle Disease Virus), and the like.

In aspect, the present disclosure is also directed to a plasmid encoding the protein conjugate.

In aspect, the present disclosure is further directed to a vaccine or other immunogenic composition comprising the protein conjugate. Treatment and vaccination methods are also contemplated herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 depicts an SDS-PAGE of purified PA-Spy0469, with an expected MW of 123,000.

FIG. 2 depicts Cryo-EM images of (D425APA63)7 using the detergent F-Fos-Choline 8, providing side-views of the heptamer. This uses 334,227 selected particles and 10,646 side-views.

FIG. 3 depicts a proposed structure of (PA63-Spy0469)7. Spy0469 (circled with dashed lines) was modeled using Alphafold available within the NCBI database (ebi.ac.uk/Tools/sss/ncbiblast/), and attached to the x-ray structure of the PA heptamer (PDB: 1TZO).

FIG. 4 depicts how anti-CMG-2 Ab competes with PAAF680 for binding (4° C.) and uptake (37° C.).

FIG. 5 depicts fractional saturation versus molar ratio of CMG-2/PA or CMG-2/PA Spy0469.

FIG. 6 shows compiled data (n=12) for Antigen-specific T cell responses induced by PA, Spy0469, PASpy0469 or SEB.

### DETAILED DESCRIPTION OF INVENTION

The ability of PA to specifically target immune cells as part of its pathology may be exploited as a platform for the development of new and better vaccines using PA as a molecular adjuvant, chaperone, or antigen delivery vehicle to target and enhance uptake and antigen presentation to immune cells, specifically to dendritic cells, resulting in enhanced activation of both CD4+ and CD8+ T cells.

The present disclosure is directed to an immunogenic protein conjugate comprising anthrax toxin component protective antigen (PA), a linker, and an antigen.

In one or more embodiments, the PA is a polypeptide encoded by the PA gene that was reported by Vodkin et al., Cloning of the protective antigen gene of Bacillus anthracis, Cell 34 693-697 (1983). The polypeptide can be at least 97%, most preferably at least 99%, and most preferably identical to wild-type PA characterized by Miller et al., Anthrax Protective Antigen. Prepore-to Pore Conversion, Biochemistry 38 (32) 10432-10441 (1999), which is incorporated by reference, or any naturally-occurring (wild type) PA polypeptide from a strain of Bacillus anthracis. The PA polypeptide may be cloned and expressed in a heterologous host such as Escherichia coli or Bacillus subtilis.

In one or more embodiments, the PA is a polypeptide that is at least 80%, preferably at least 90%, more preferably at least 95%, still more preferably at least 97%, or most preferably at least 99% identical to a wild type PA sequence of Bacillus anthracis (SEQ ID NO:1) and conserved functional variants thereof reported in the literature, which may have one or two amino acid residues different from SEQ ID NO:1. Preferably the PA is a polypeptide which comprises each of the four PA domains (SEQ ID NO:2-5). The reference sequence for PA is UniProt: Swiss-Prot: P13423 (see also GenBank AF306778), which are each incorporated by reference herein. In one or more embodiments, the PA sequence is encoded by a plasmid having SEQ ID NO: 6. The expressed PA may thus further comprise a periplasmic signal sequence SEQ ID NO:7 (MKKRKVLIPLMALSTILVSSTGNLEVIQA), or periplasmic signal sequence SEQ ID NO:17 (MKYLLPTAAAGLLLLAAQPAMA), or an in frame cloning sequence MDIGINSDPM (SEQ ID NO: 18).

It is understood that homologs and analogs have the characteristics of the anthrax PA described herein and may be used in the methods of the invention, provided they include all four domains of PA. The term also includes any recombinant B. anthracis PA, or other modified forms (variants) that retain the ability to bind dendritic cells (specifically for binding CMG-2 on dendritic cells).

The antigen can comprise any antigen comprising an epitope for inducing an immune response against an invasive, pathogenic, or infectious disease or condition, such as an antigen or epitope derived from a virus, bacteria, fungus, cancer, or the like. Examples include an antigen from a group A Streptococcus species, Mycobacterium tuberculosis, a malaria-causing Plasmodium species, Human immunodeficiency virus (HIV), human cytomegalovirus (CMV), or any other antigen. In certain embodiments, the antigen can comprise an antigen from Streptococcus pyogenes. The antigen can comprise Spy0469 comprising an amino acid sequence of SEQ ID NO: 20 or 21 or Spy 1228 comprising an amino acid sequence of SEQ ID NO: 25. In certain embodiments, the antigen can comprise SEQ ID NO: 22, SEQ ID NO: 23, SEQ ID NO: 24, or a combination thereof. Each of SEQ ID NO: 22-24 are key epitopes from Spy0469. Other suitable antigens that can be conjugated to the PA include pp65 (antigen from human cytomegalovirus), PsaA (antigen from Streptococcus pneumoniae), SpyTag003-OspC (antigen from Borrelia burgdorferi; Lyme Disease), NDV HN-SpyTag (antigen from Newcastle Disease Virus), and the like. Non-limiting examples of antigens for specific conditions and diseases are described in the tables below.

TB Antigens

| | | Suitability | |
|---|---|---|---|
| | | for PA | Medical |
| | | conjugate | Importance & |
| Antigen | Description | vaccine | Vaccine Gap |
| ESAT-6 | Secreted | Used in fusion | Key in latent |
| (Early | protein | proteins (e.g., | TB; no effective |
| Secreted | involved in | with CFP-10); PA | adult vaccine; |
| Antigenic | virulence; | fusion could | part of |
| Target 6) | highly | improve | diagnostic |
| | immunogenic, | cytosolic | tools but |
| | induces | delivery for | untapped for |
| | strong T-cell | MHC presentation, | protection. |
| | responses. | similar to | |
| | | anthrax toxin | |
| | | mechanisms. | |
| Ag85B | Surface | Recombinant | High burden |
| (Mycolyl | enzyme in | forms elicit | in low- |
| transferase) | cell wall | CD4+/CD8+ | resource areas; |
| | biosynthesis; | responses; | included in |
| | conserved and | PA could | M72/AS01E |
| | protective | enhance DC | candidate |
| | in animal | uptake, as | (49.7% efficacy |
| | models. | seen in other | in trials), but |
| | | bacterial | no licensed |
| | | antigen-PA | alternative |
| | | studies. | to BCG. |
| HtpG (Heat | Chaperone-like | Multi-domain | Targets |
| shock | protein aiding | structure | drug-resistant |
| protein) | pathogenesis; | suitable | TB; no vaccine |
| | induces robust | for fusion; | covers all |
| | immunity in | promising | strains |
| | preclinical | for latency- | effectively. |
| | studies. | associated | |
| | | antigens, with | |
| | | PA targeting | |
| | | to boost | |
| | | T-cell priming. | |

HIV Antigens

| | | Suitability | Medical |
|---|---|---|---|
| | | for PA | Importance |
| | | conjugate | & Vaccine |
| Antigen | Description | vaccine | Gap |
| gp120 | Surface | Stabilized | Core of |
| (Envelope | protein | trimers | most HIV |
| glycoprotein) | mediating | used in | candidates; |
| | viral entry; | candidates; PA | ongoing |
| | elicits bNAbs | could direct | trials (e.g., |
| | in trials. | to DCs for | mRNA-Env) |
| | | better bNAb | show promise |
| | | induction, | but no |
| | | as in | approval. |
| | | nanoparticle-Env | |
| | | fusions. | |
| Gag (Group- | Core structural | Expressed in | Controls viral |
| specific | protein; | empirical | replication; |
| antigen) | conserved, | vaccines; PA | no vaccine |
| | induces | fusion enhances | despite |
| | CD8+ T-cells. | cytosolic | decades of |
| | | access for | research. |
| | | CTL responses, | |
| | | akin to | |
| | | anthrax-based | |
| | | HIV strategies. | |
| Retanef | Regulatory | Pre-made | Addresses |
| (Fusion | proteins | fusion ideal | immune |
| of Rev, | expressed | for PA | evasion; |
| Tat, Nef) | early; | conjugation; | experimental |
| | chimeric | targets | but critical |
| | antigen for | early infection | for prevention. |
| | broad | stages, boosting | |
| | coverage. | multi-epitope | |
| | | responses. | |

Group A strep antigens

| | | Suitability | |
|---|---|---|---|
| | | for PA | Medical |
| | | conjugate | Importance & |
| Antigen | Description | vaccine | Vaccine Gap |
| SpyCEP | Surface protease | Elicits antibodies | Prevents |
| (Streptococcal | cleaving IL-8; | in mouse models; | neutrophil |
| protease) | highly | PA could improve | evasion; no |
| | conserved and | DC targeting for | vaccine, |
| | protective. | opsonization, | despite |
| | | similar to | rheumatic |
| | | Spy0469. | fever burden. |
| C5a Peptidase | Enzyme inactivating | Broadly | High |
| (ScpA) | complement; | protective; | invasiveness |
| | part of | fusion to | in children; |
| | multi-antigen | PA enhances | candidates |
| | candidates | immunogenicity | in preclinical |
| | like Spy7. | without | but no human |
| | | adjuvants. | vaccine. |
| Spy AD | Surface adhesin; | Identified | |
| (Adhesin) | conserved, induces | via antigenome; | |
| | protection in | | |
| | models. | | |

Breast, Ovarian, Gastric cancers

| | | Suitability | |
|---|---|---|---|
| | | for PA | Medical |
| | | conjugate | Importance & |
| Antigen | Description | vaccine | Vaccine Gap |
| HER2/neu | Transmembrane | Fused | ~232,000 new |
| (Human | protein with | domains (e.g., | US breast |
| Epidermal | extracellular | ICD or ECD) | cases yearly; |
| Growth | and | used in DC | no licensed |
| Factor | intracellular | vaccines; PA | vaccine despite |
| Receptor 2) | domains; | enhances | trials (e.g., |
| | elicits CD8+ | cytosolic | ES2B-C001, αDC1); |
| | T-cells and | delivery | enhances |
| | antibodies | for cross- | response to |
| | in trials. | priming, | therapies like |
| | | synergizing | Enhertu. |
| | | with existing | |
| | | HER2-DC1 | |
| | | approaches. | |
| MUC1 | Glycoprotein | Variable | Expressed in |
| (Mucin 1) | aberrantly | number tandem | >90% of |
| | glycosylated | repeat (VNTR) | breast/pancreatic |
| | in tumors; | domain | cancers; |
| | induces | suitable for | no licensed |
| | humoral and | fusion; PA | vaccine, trials |
| | cellular | could | ongoing (e.g., |
| | immunity. | improve DC | mRNA-based). |
| | | uptake | |
| | | and break | |
| | | tolerance in | |
| | | epithelial | |
| | | cancers. | |

Melanoma and lung cancers

| | | Suitability | |
|---|---|---|---|
| | | for PA | Medical |
| | | conjugate | Importance & |
| Antigen | Description | vaccine | Vaccine Gap |
| MAGE-A3 | Intracellular | Used in peptide/DC | In 40-50% of |
| (Melanoma- | protein; highly | vaccines; PA fusion | melanomas/non- |
| Associated | immunogenic, | aids cytosolic | small cell |
| Antigen A3) | induces | access | lung cancers; |
| | CD8+ T-cells | for MHC I | no licensed |
| | in trials. | presentation, | vaccine after |
| | | addressing | failed Phase |
| | | past trial | III, but new |
| | | limitations. | platforms |
| | | | promising. |
| NY-ESO-1 | CTA expressed | Full-length or | In melanomas, |
| (New York | in multiple | epitopes | sarcomas |
| Esophageal | cancers; | fused in | (~30-40%); no |
| Squamous | elicits | trials; PA | licensed |
| Cell | strong | enhances DC | vaccine, Phase |
| Carcinoma-1) | T-cell | targeting | I/II trials |
| | responses. | for better | ongoing. |
| | | anti-tumor | |
| | | efficacy. | |

Solid tumors and leukemia

| | | Suitability | |
|---|---|---|---|
| | | for PA | Medical |
| | | conjugate | Importance & |
| Antigen | Description | vaccine | Vaccine Gap |
| WT1 | Transcription | Peptide-based | In AML, |
| (Wilms' | factor | in DC | ovarian |
| Tumor 1) | overexpressed | vaccines; | (~70-90%); |
| | in cancers; | PA fusion | no licensed |
| | HLA-restricted | improves | vaccine, |
| | epitopes | intracellular | trials show |
| | induce CTLs. | delivery, | remission |
| | | boosting | prolongation. |
| | | T-cell | |
| | | priming. | |
| p53 | Mutated | Used in | Mutated |
| (Tumor | guardian | multi-epitope | in 50% of |
| Protein | protein; | vaccines; | cancers; no |
| 53) | conserved | PA could | licensed |
| | epitopes | overcome | vaccine, |
| | for broad | self-tolerance | potential for |
| | coverage. | via DC | universal |
| | | targeting. | application. |

The antigen can be selected by the following process. First, antigens are selected that display increased gene expression during interaction with the host, as these are likely to represent key factors in the establishment of an infection. From the upregulated genes, a subset is selected that are i) conserved with over 90% identity in most of the known genomes of the targeted organism and ii) predicted to have an extracellular location. The selected antigens are then expressed as recombinant proteins. Each antigen is tested for proper structure and stability using circular dichroism, IgG recognition, including specific epitopes, and recognition by T cells, particularly IFNγ response. Mice are then immunized with the most promising antigens and tested for generating an immune response and inducing protection.

The antigen is conjugated at the C-terminus of PA via a linker or linker system. The linker can comprise any amino acid-based linker known in the art, and particularly are preferred as peptide-based linkers of 10 amino acid residues or less (e.g., peptide linkers consisting of from 2 to 10 amino acid residues). In one or more embodiments, the linkers comprise glycine (G) and/or serine(S) residues, and may further include 1 to 2 glutamic acid linkers (E). Gly/Ser linkers show a good balance of flexibility, solubility, and protease resistance while Glu in the linker increases hydrophilicity. Other suitable amino acid residues include threonine. In general, the linker is designed to be resistant to enzymatic degradation or cleavage (particularly protease cleavage), and therefore non-cleavable amino acid pairs are selected. The amino acid residues should also be selected to introduce stearic hindrance and promote elongation of the conjugate to avoid the PA and antigen from folding back onto each other. The link is preferably free of cleavable lysine (K) or arginine (R) linkers. The linker should also be designed to reduce the chance of interference in the structure or function of the fused protein, and specifically to avoid interference with the ability of PA to initiate initial binding to the target dendritic cell. Non-limiting example of peptide linkers include SST, GSG, STTA (SEQ ID NO:30) GSGESGSG (SEQ ID NO:9), GSGEESGSG (SEQ ID NO: 10), GSGESGSGS (SEQ ID NO:11), GSGEESGSGS (SEQ ID NO:12), and GGGGSGGGGS (SEQ ID NO:13).

In certain embodiments, the linker can comprise a binding pair system, such as a system comprising a SpyTag peptide and a SpyCatcher peptide. The antigen of interest can be conjugated to a first member of a specific binding pair, such as SpyTag, and PA can be bound via its C-terminus (domain 4) to a second cognate member of a specific binding pair, such as SpyCatcher or fragments/derivatives thereof. The SpyTag and SpyCatcher are then covalently bound to each other to complete the protein conjugate, and specifically to generate a fusion protein. Thus, the linker comprised of a “specific binding pair” refers to a first polypeptide, and a second cognate member, such as a second polypeptide, that interact to form a covalent isopeptide bond under conditions that enable or facilitate isopeptide bond formation, wherein the term “cognate” refers to components that function together by to reacting together to form an isopeptide bond. Specific binding pairs capable of interacting to form a covalent isopeptide bond are reviewed in Veggiani et al. (2014) Trends Biotechnol. 32:506, and include, for example, peptide: peptide binding pairs such as SpyTag: SpyCatcher, SpyTag002: SpyCatcher002, SpyTag: KTag, isopeptag: pilin C, SnoopTag: SnoopCatcher and others. Spy Tag002: SpyCatcher002 and SpyTag003: SpyCatcher003 are different iterations of Spy Tag: Spy Catcher.

The SpyTag can comprise an amino acid sequence of
(SEQ ID NO: 14)
AHIVMVDAYKPT.
The SpyCatcher peptide can comprise an amino acid sequence of
(SEQ ID NO: 15)
MSYYHHHHHHDYDIPTTENLYFQGAMVDTLSGLSSEQGQSGDMTIEEDSATHIKFSKRD
EDGKELAGATMELRDSSGKTISTWISDGQVKDFYLYPGKYTFVETAAPDGYEVATAITF
TVNEQGQVTVNGKATKGDAHI.
The SpyCatcher peptide can comprise an amino acid sequence of
(SEQ ID NO: 26)
SATHI KFSKRDEDGR ELAGATMELR DSSGKTISTW
ISDGHVKDFY LYPGKYTFVE TAAPDGYEVA TPIEFTVNED GQVTV .
The SpyCatcher peptide can comprise an amino acid sequence of
(SEQ ID NO: 27)
MVTTLSGLSGEQGPSGDMTTEEDSATHIKFSKRDEDGRELAGATMELRDSSGKTISTWIS
DGHVKDFYLYPGKYTFVETAAPDGYEVATPIEFTVNEDGQVTVDGEATEGDAHT.

The linker can preferably be attached to domain 4 of PA. Use of isopeptide binding partner pairs, such as SpyCatcher and SpyTag, was pioneered in Streptococcus pyogenes and is described in detail in U.S. Pat. No. 9,547,003, issued Jan. 17, 2017, incorporated by reference herein, based upon attachment proteins from a bacterium, has been established as a technology to irreversibly and spontaneously conjugate recombinant proteins and the like under physiological conditions. The fibronectin binding protein, FbaB, from Streptococcus pyogenes contains a CnaB2 adhesin domain. CnaB2 is stabilized by a spontaneous reaction of Lys and Asp side chains to form an isopeptide bond. CnaB2 has been split into the 13-residue SpyTag peptide and the 116-residue SpyCatcher protein, that each may be fused to two entities that it is desired to bind via an isopeptide bond. Various Catcher and Tag pairs are now available, some based upon modifications of SpyCatcher and SpyTag, and others based upon similar chemistry from alternative bacterial proteins.

Other binding pair systems similar to SpyTag and SpyCatcher include DogTag and DogCatcher, DogTag and SnoopTag, DogTag and SnoopTagJr, SnoopTag and SnoopCatcher, or variants thereof. Other suitable binding partner pairs will be known to the skilled person. One can also use a linker between SpyTag or SpyCatcher and the protein/antigen of interest. The linker increases the accessibility of SpyTag/SpyCatcher for faster and higher yielding reactions.

The protein conjugate has a higher affinity for CMG-2 than the antigen alone. In one or more embodiments, the protein conjugate can comprise or consist of:

MKYLLPTAAA GLLLLAAQPA MAMDIGINSD PMEVKQENRL LNESESSSQG
LLGYYFSDLN FQAPMVVTSS TTGDLSIPSS ELENIPSENQ YFQSAIWSGF IKVKKSDEYT
FATSADNHVT MWVDDQEVIN KASNSNKIRL EKGRLYQIKI QYQRENPTEK
GLDFKLYWTD SQNKKEVISS DNLQLPELKQ KSSNSRKKRS TSAGPTVPDR
DNDGIPDSLE VEGYTVDVKN KRTFLSPWIS NIHEKKGLTK YKSSPEKWST
ASDPYSDFEK VTGRIDKNVS PEARHPLVAA YPIVHVDMEN IILSKNEDQS
TQNTDSQTRT ISKNTSTSRT HTSEVHGNAE VHASFFDIGG SVSAGFSNSN SSTVAIDHSL
SLAGERTWAE TMGLNTADTA RLNANIRYVN TGTAPIYNVL PTTSLVLGKN
QTLATIKAKE NQLSQILAPN NYYPSKNLAP IALNAQDDFS STPITMNYNQ
FLELEKTKQL RLDTDQVYGN IATYNFENGR VRVDTGSNWS EVLPQIQETT
ARIIFNGKDL NLVERRIAAV NPSDPLETTK PDMTLKEALK IAFGFNEPNG
NLQYQGKDIT EFDFNFDQQT SQNIKNQLAE LNATNIYTVL DKIKLNAKMN
ILIRDKRFHY DRNNIAVGAD ESVVKEAHRE VINSSTEGLL LNIDKDIRKI LSGYIVEIED
TEGLKEVIND RYDMLNISSL RQDGKTFIDF KKYNDKLPLY ISNPNYKVNV
YAVTKENTII NPSENGDTST NGIKKILIFS KKGYEIG SST AQAQEWT PRSVTEIKS
ELVLVDNVFT YTVKYGDTLS TIAEAMGIDV HVLGDINHIA NIDLIFPDTI
LTANYNQHGQ ATNLTVQAPA SSPASVSHVP SSEPLPQASA TSQPTVPMAP
PATPSDVPTT PFASAKPDSS VTASSELTSS TNDVSTELSS ESQKQPEVPQ EAVPTPKAAE
TTEVEPKTDI SEAPTSANRP VPNESASEEV SSAAPAQAPA EKEETSAPAA
QKAVADTTSV ATSNGLSYAP NHAYNPMNAG LQPQTAAFKE EVASAFGITS
FSGYRPGDPG DHGKGLAIDF MVPENSALGD QVAQYAIDHM AERGISYVIW
KQRFYAPFAS IYGPAYTWNP MPDRGSITEN HYDHVHVSFN A (PA-Spy0469 conjugate
(SEQ ID NO: 19)).

In one or more embodiments, the protein conjugate can comprise or consist of:

(SEQ ID NO: 28)
MKYLLPTAAA GLLLLAAQPA MAMDIGINSD PMEVKQENRL LNESESSSQG
LLGYYFSDLN FQAPMVVTSS TTGDLSIPSS ELENIPSENQ YFQSAIWSGF
IKVKKSDEYT FATSADNHVT MWVDDQEVIN KASNSNKIRL EKGRLYQIKI
QYQRENPTEK GLDFKLYWTD SQNKKEVISS DNLQLPELKQ KSSNSRKKRS
TSAGPTVPDR DNDGIPDSLE VEGYTVDVKN KRTFLSPWIS NIHEKKGLTK
YKSSPEKWST ASDPYSDFEK VTGRIDKNVS PEARHPLVAA YPIVHVDMEN
IILSKNEDQS TQNTDSQTRT ISKNTSTSRT HTSEVHGNAE VHASFFDIGG
SVSAGFSNSN SSTVAIDHSL SLAGERTWAE TMGLNTADTA RLNANIRYVN
TGTAPIYNVL PTTSLVLGKN QTLATIKAKE NQLSQILAPN NYYPSKNLAP
IALNAQDDFS STPITMNYNQ FLELEKTKQL RLDTDQVYGN IATYNFENGR
VRVDTGSNWS EVLPQIQETT ARIIFNGKDL NLVERRIAAV NPSDPLETTK
PDMTLKEALK IAFGFNEPNG NLQYQGKDIT EFDFNFDQQT SQNIKNQLAE
LNATNIYTVL DKIKLNAKMN ILIRDKRFHY DRNNIAVGAD ESVVKEAHRE
VINSSTEGLL LNIDKDIRKI LSGYIVEIED TEGLKEVIND RYDMLNISSL
RQDGKTFIDF KKYNDKLPLY ISNPNYKVNV YAVTKENTII NPSENGDTST
NGIKKILIFSKKGYEIGGSGESGSGSMVTTLSGLSGEQGPSGDMTTEEDSATHIKFSKRDE
DGRELAGATMELRDSSGKTISTWISDGHVKDFYLYPGKYTFVETAAPDGYEVATPIEFT
VNEDGQVTVDGEATEGDAHT PA-SpyCatcher003 conjugate, to which an
antigen can be bound via a SpyTag cognate binding partner.

In one or more embodiments, the protein conjugate can comprise or consist of:

(SEQ ID NO: 29)
MKYLLPTAAA GLLLLAAQPA MAMDIGINSD PMEVKQENRL LNESESSSQG
LLGYYFSDLN FQAPMVVTSS TTGDLSIPSS ELENIPSENQ YFQSAIWSGF
IKVKKSDEYT FATSADNHVT MWVDDQEVIN KASNSNKIRL EKGRLYQIKI
QYQRENPTEK GLDFKLYWTD SQNKKEVISS DNLQLPELKQ KSSNSRKKRS
TSAGPTVPDR DNDGIPDSLE VEGYTVDVKN KRTFLSPWIS NIHEKKGLTK
YKSSPEKWST ASDPYSDFEK VTGRIDKNVS PEARHPLVAA YPIVHVDMEN
IILSKNEDQS TQNTDSQTRT ISKNTSTSRT HTSEVHGNAE VHASFFDIGG
SVSAGFSNSN SSTVAIDHSL SLAGERTWAE TMGLNTADTA RLNANIRYVN
TGTAPIYNVL PTTSLVLGKN QTLATIKAKE NQLSQILAPN NYYPSKNLAP
IALNAQDDFS STPITMNYNQ FLELEKTKQL RLDTDQVYGN IATYNFENGR
VRVDTGSNWS EVLPQIQETT ARIIFNGKDL NLVERRIAAV NPSDPLETTK
PDMTLKEALK IAFGFNEPNG NLQYQGKDIT EFDFNFDQQT SQNIKNQLAE
LNATNIYTVL DKIKLNAKMN ILIRDKRFHY DRNNIAVGAD ESVVKEAHRE
VINSSTEGLL LNIDKDIRKI LSGYIVEIED TEGLKEVIND RYDMLNISSL
RQDGKTFIDF KKYNDKLPLY ISNPNYKVNV YAVTKENTII NPSENGDTST
NGIKKILIFS KKGYEIGGSG ESGSGSATHI KFSKRDEDGR ELAGATMELR
DSSGKTISTW ISDGHVKDFY LYPGKYTFVE TAAPDGYEVA TPIEFTVNED
GQVTV PA-SpyCatcher003 conjugate,, to which an antigen can be
bound via a SpyTag cognate binding partner.

The present disclosure is also directed to a vector or plasmid encoding the protein conjugate described herein. In one or more embodiments, the vector or plasmid comprises or consists of SEQ ID NO:6.

The present disclosure is further directed to a vaccine or immunogenic composition comprising the protein conjugate. Those skilled in the art will appreciate that depending on the intended mode of administration, the protein conjugate can be in various pharmaceutical compositions. The compositions will comprise the protein conjugate in a therapeutically effective amount in combination with a pharmaceutically acceptable carrier and, in addition, may include other medicinal agents, pharmaceutical agents, carriers, adjuvants (immunopotentiating agents), diluents, etc. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would naturally be selected to minimize any degradation of the compound or other agents and to minimize any adverse side effects in the subject, as would be well known to one of skill in the art.

In one or more embodiments, the immunogenic compositions of the present invention may be formulated by dispersing the protein conjugate in the desired amount in any pharmaceutical carrier suitable for use in vaccines. Any pharmaceutical carrier suitable for administration to mammals which does not interfere with the immunogenicity of the protein conjugate may be employed. Exemplary carriers include aqueous solutions such as normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO) or other acceptable vehicles, and the like.

The vaccine will induce an immune response against the antigen included in the conjugate. In one or more embodiments, the vaccine can be a group A Streptococcus vaccine. The vaccine can be a Streptococcus pyogenes vaccine.

Methods for therapeutic and prophylactic use of the immunogenic protein conjugates are also contemplated herein. In one aspect, a method for inducing an immunogenic response in a subject is described, which comprises administering to the subject a therapeutically-effective amount of the protein conjugate to a subject in need thereof. As used herein, a “therapeutically-effective” amount refers to the amount that will elicit the biological or medical response of a tissue, system, or subject that is being sought by a researcher or clinician, and in particular elicit some desired therapeutic or prophylactic effect as against the anthrax infection by preventing and/or inhibiting toxin activity and/or pathogenesis of the toxin (e.g., through infiltration across the cell membrane in the subject). One of skill in the art recognizes that an amount may be considered therapeutically “effective” even if the condition is not totally eradicated or prevented, but it or its symptoms and/or effects are improved or alleviated partially in the subject.

The vaccine can be administered as with any other protein vaccine, including either subcutaneous (SC) or intramuscular (IM) or intradermal (ID), intranasal or intravenous. The immunogenic protein conjugates according to the embodiments disclosed herein are useful in treating and/or preventing infections or other diseases by eliciting an immune response against an antigenic or pathogenic agent in a subject. Thus, embodiments described herein have broad-spectrum therapeutic and/or prophylactic uses. The terms “therapeutic” or “treat,” as used herein, refer to treatments that are intended to produce a beneficial change in an existing condition (e.g., infection, disease, disorder) of a subject, such as by reducing the severity of the clinical symptoms and/or effects of the infection, and/or reducing the duration of the infection/symptoms/effects in the subject (who has already been infected). The terms “prophylactic” or “prevent,” as used herein, refer to treatments that are intended to inhibit or ameliorate the effects of a future infection or disease to which a subject may be exposed (but is not known to be currently infected with). In some cases, the immunogenic protein conjugates may prevent the development of observable morbidity from infection (i.e., near 100% prevention). In other cases, the immunogenic protein conjugates may only partially prevent and/or lessen the extent of morbidity due to the infection (i.e., reduce the severity of the symptoms and/or effects of the infection, and/or reduce the duration of the infection/symptoms/effects). In either case, the immunogenic protein conjugates are still considered to “prevent” the target infection or disease, in the context of the present application.

In one or more embodiments, immunogenic protein conjugates according to the invention can be used to induce protective immunity in the subject. The term “immunogenic” refers to the ability of the protein conjugates to provoke an immune response in the subject after administration, and in particular against the antigen contained in the conjugate. The terms “protection” or “protective immunity” refers herein to the ability of the serum antibodies and cellular response induced during immunization to protect (partially or totally) against infection and/or pathogenesis by an antigenic or pathogenic agent. Thus, a subject immunized by the protein conjugates of the invention will experience limited growth and spread of an antigenic or pathogenic virus or microorganism compared to an unvaccinated control. This is because the immune response of the subject will be enhanced against the pathogen due to development of antibodies against the antigen contained in the conjugate. The activation of the immune response, and specifically T cells is enhanced by the selective targeting of the immune cells by the PA component of the conjugate, and transport of the antigen to the cells.

The disclosed embodiments are suitable for various routes of administration, depending upon the particular carrier and other ingredients used. For example, the prophylactic and/or therapeutic conjugates or compositions thereof can be injected intramuscularly (IM), subcutaneously (SC), intradermally (IV), or intravenously. They can also be administered via mucosa such as intranasally or orally. In some embodiments, the conjugates or compositions thereof can be provided in unit dosage form in a suitable container. The term “unit dosage form” refers to a physically discrete unit suitable as a unitary dosage for human or animal use. Each unit dosage form may contain a predetermined amount of the protein conjugate (and/or other active agents) in the carrier calculated to produce a desired effect. The invention also relates to kits for vaccinating mammals for the treatment or prevention of infection in a mammal due to an antigenic agent, comprising the immunogenic conjugates of the invention. The conjugates, compositions, and methods can be used for priming doses, prime and boost regimens, or for boosting immunity in an already infected individual.

Experiments indicate that attachment of the Spy0469 antigen (SEQ ID NO:21) to domain 4 of PA (the receptor binding domain, SEQ ID NO: 5) does not disrupt the ability of PA to bind to the host cellular receptor CMG-2. This suggests that other vaccine antigens may be attached to domain 4 of PA, which can then target to dendritic cells for antigen presentation and enhancement of the immune response against the antigenic agent. Based on previous published findings that immune responses to PA pre-bound to CMG-2 lowers the immune response in mice, it is suggested that PA binding to CMG-2 present on immune cells is a necessary prerequisite to an effective immune response to PA.

The properties of a conjugate between PA and Spy0469, a surface protein of Streptococcus pyogenes that has been shown to induce protective immunity in mouse models of infection were investigated. Because of the high frequency of T cells to epitopes of Spy0469 due to high incidence of S. pyogenes infections in childhood, the ability of human PBMCs and isolated DC subsets to present T cell epitopes of Spy0469 was examined. This conjugate was used to determine binding to human PBMC cell populations and for T-cell activation. Further, it is intended to evaluate the PA-Spy0469 as a potential vaccine to protect against S. pyogenes infection. Hence, the structural analysis of the hybrid protein, the heptameric (PA₆₃-Spy0469)₇ conjugate, is a high priority.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used in this application, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

As used in this application, including the appended claims, the singular forms “a,” “an,” and “the” include plural references unless the content clearly dictates otherwise, and are used interchangeably with “at least one” and “one or more.”

As used herein, “percent identity” between amino acid or nucleic acid sequences is synonymous with “percent homology,” which can be determined using the algorithm of Karlin and Altschul (Proc. Natl. Acad. Sci. USA 87 2264-2268 (1990)), modified by Karlin and Altschul (Proc. Natl. Acad. Sci. USA 90 5873-5877, (1993)). Such an algorithm is incorporated into the NBLAST and XBLAST programs of Altschul et al., Basic Local Alignment Search Tool, J. Mol. Biol. 215 403-410, (1990). BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide (e.g., SEQ ID NO. 1). To obtain gapped alignments for comparison purposes, Gapped BLAST is utilized as described in Altschul et al., Gapped BLAST and PSI-BLAST: A new generation of protein database search programs, Nucleic Acids Res. 25 3389-3402 (1997). When utilizing BLAST and Gapped BLAST programs, the default parameters of the respective programs (e.g., XBLAST and NBLAST) are used which are available from the National Institutes of Health. A variant can also include, e.g., an internal deletion or insertion, a conservative or non-conservative substitution, or a combination of these variations from the sequence presented. In any event, it will be appreciated that fragments of the sequences described herein must remain “functional” as compared to the original sequence. Thus, the term “functional fragment” refers to sequences that may include insertion, deletions, and the like, but which nonetheless comprise or encode for a protein that retains the functionality of the original sequence (e.g., original binding specificity or ability, etc.).

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

The invention will be further described in the following examples, which do not limit the scope of the invention described in the claims.

### EXAMPLES

The following examples set forth methods in accordance with the invention. It is to be understood, however, that these examples are provided by way of illustration and nothing therein should be taken as a limitation upon the overall scope of the invention.

### Example 1: (PA₆₃-Spy0469)₇ Conjugate Structure

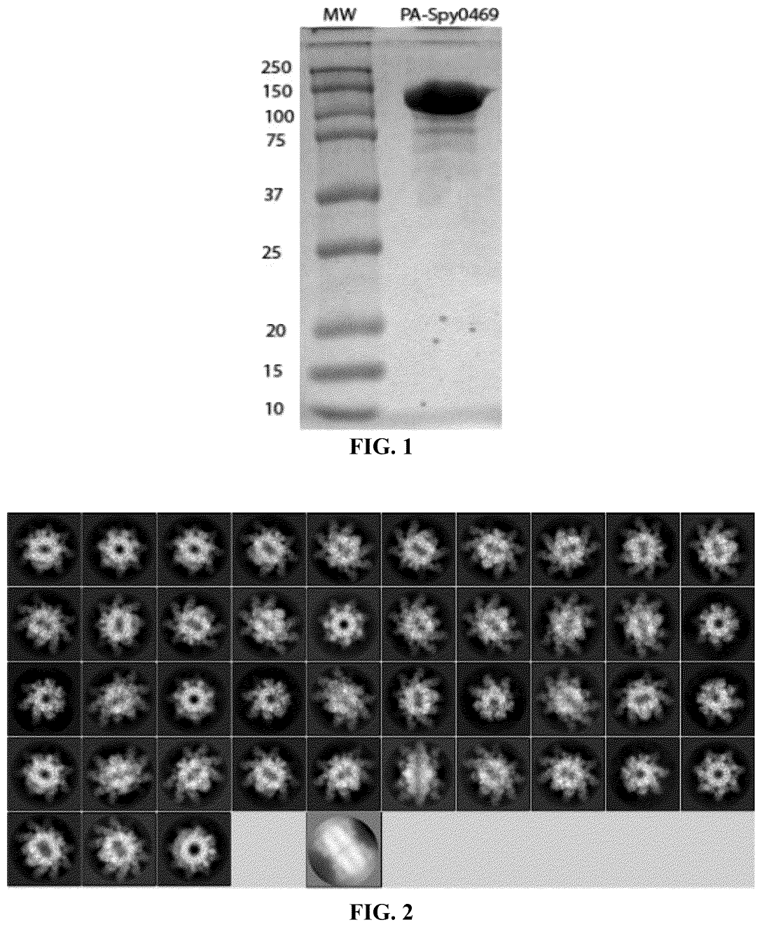

To determine the structure of the (PA₆₃-Spy0469)₇ conjugate, the bacterially expressed protein was purified to homogeneity (FIG. 1). Recently, the structure of the PA heptamer (D425A, at low pH) was determined, demonstrating the feasibility of studying the heptameric structure. In this case, the prepore complex (8.43 μM-pH 8.25) was diluted 1:3 with 50 mM Tris, 25 mM MES, 25 mM Acetate and 200 mM NaCl buffer (pH 5.0) and 1 μl 900 μM F-FOS-Choline 8 (in the same buffer), which allowed for both top-down and side views. Holey carbon grids (Quantifoil Cu 300 mesh R1.2/1.3) were loaded with 3.5 μL sample and plunge frozen in liquid nitrogen cooled liquid ethane using a vitrobot (FEI) with a blotforce of −10, for 3.5 seconds at 100% humidity and 4° C. In one set of conditions, the purified prepore complex will be diluted into 50 mM Tris, 25 mM MES, 25 mM Acetate and 200 mM NaCl buffer (pH 8.0), and in another the same conditions except with the addition of F-Fos-Choline 8 detergent. Addition of this detergent should allow for side views which in turn are needed for a full structure determination (FIG. 2). However, since this is a previously uncharacterized complex, these conditions may not necessarily be favorable for forming side views of the complex, and will rely on local expertise to provide suggestions on how to achieve side-views.

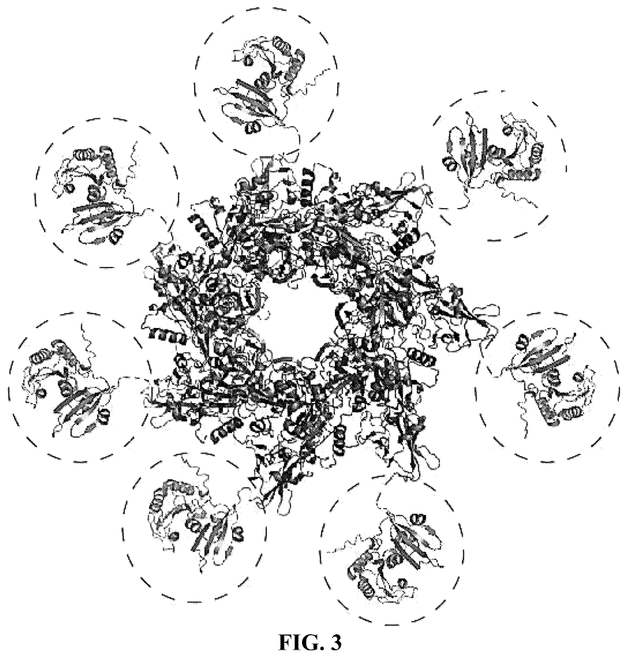

These experiments will be used to determine a three-dimensional structure of the PA-Spy0469 complex, which is anticipated to give rise to a flower-like structure (FIG. 3). The goal is to screen similar conditions and collect data sets with and without the addition of F-Fos-Choline 8.

### Example 2: In Vitro Study of Anthrax Protective Antigen and Spy0469 Conjugate (PA-Spy0469)

The anthrax protective antigen and Spy0469 conugate (PA-Spy0469) was made using spliced-overlap extension PCR (Morrison and Desrosiers (1993) Biotechniques vol 14, 454-457), using the Spy0469 sequence from Addgene and the PA sequence from the pQE80PA plasmid. A gating strategy was used for antigen-specific T cells (not shown), and SEB stimulated groups were used as a control. Antigen-specific T cells were induced by PA, Spy0469, PASpy0469, or SEB. The PASpy0469 stimulated group showed slightly higher antigen-specific T cells, especially CD4+ T cells, compared to the PA and Spy0469 alone stimulated groups, but without statistical difference due to the sample size (n=2). PPA (10 ug/mL), Spy0469 (5 ug/mL) and PASpy0469 (15 ug/mL) were added with similar molar concentration. All the data were subtracted with unstimulated control to eliminate the background. SEB indicates Staphylococcal Enterotoxin B, as positive control.

### Example 3: In Vitro Study of Anthrax Protective Antigen Vaccine Binding with Capillary Morphogenesis Protein 2

Experiments were conducted to assess how anti-CMG-2 Ab binds cells at 4° C. The following treatment groups were subjected to these conditions:
1) Anti-CMG-2 Ab was added at 2.375 ug/ml. Then, 30 minutes later, L/D was added for 5 minutes. Then 5 μl of AF647 (APC) goat anti-mouse IgG secondary Ab was added for 20 minutes. This was all done in PBS with 1% PFA.
2) Isotype (control mouse IgG1) Ab was added at 2.375 ug/ml. Then, 30 minutes later, L/D was added for 5 minutes. Then 5 μl of AF647 (APC) goat anti-mouse IgG secondary Ab was added for 20 minutes. This was all done in PBS with 1% PFA.
3) Unstimulated
4) 2 μl of anti-human CD45 Ab (HI30) was added as a positive control. Then, 30 minutes later, L/D was added for 5 minutes. Then 5 μl of AF647 (APC) goat anti-mouse IgG secondary Ab was added for 20 minutes. This was all done in PBS with 1% PFA. The percentage of APC+ cells in live cells were background subtracted by the unstimulated group. For the “AF647 (APC) goat anti-mouse IgG (H+L) secondary Ab (#A-21236)” goat anti-mouse secondary Abs, the background was very high even in the unstimulated group. Isotype indicates isotype control mouse IgG1. Anti-CD45 was used as the positive control. (data not shown)

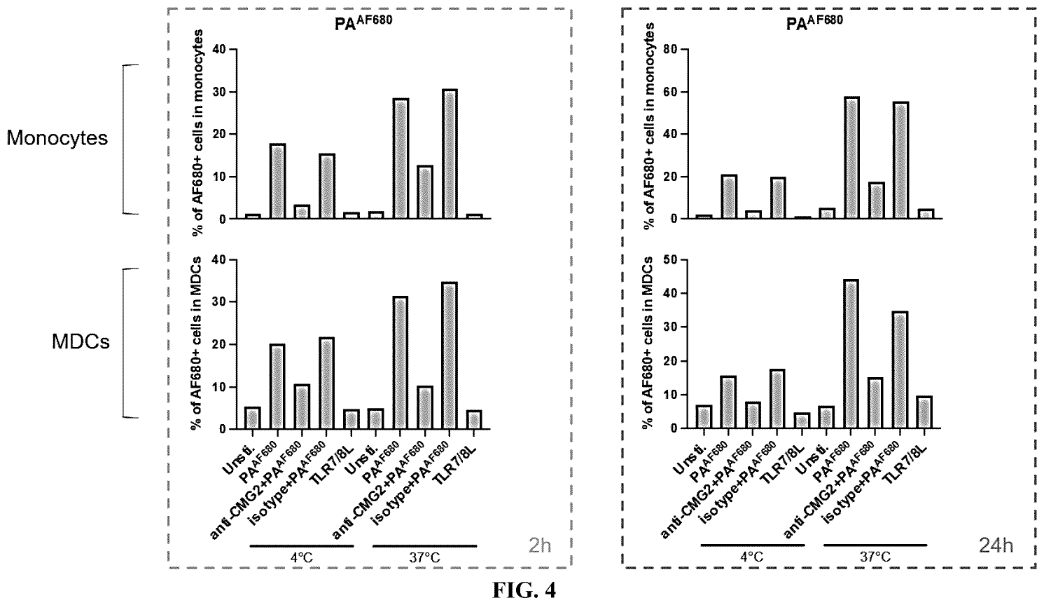

Experiments were also conducted to assess how anti-CMG-2 Ab competes with PAAF680 for binding (4° C.) and uptake (37° C.). The results are shown in FIG. 4. The following treatment groups were subjected to the 2-hour and 24-hour conditions for monocytes and MDCs:
1) 1 ug/ml PAAF680 was added.
2) 2.375 ug/ml anti-CMG-2 Ab was added. Then, 30 minutes later, 1 μg/ml PAAF680 was added.
3) 2.375 ug/ml Isotype (control mouse IgG1) Ab was added. Then, 30 minutes later, 1 μg/ml PAAF680 was added.
4) Unstimulated.
5) 5 ug/ml TLR 7/8L.

It was also found that anti-CMG-2 Ab competes with PAAF680 for binding (4° C.) and uptake (37° C.) when PBMCs were incubated for 2 h (histograms not shown) and 24 h (histograms not shown). It was also found that Anti-CMG-2 Ab competes with PAAF680 but does not (or slightly) affect CD80 and CCR7 when PBMCs were incubated for 2 h and 24 h.

### Example 4: PA and PASpy0469 Binding to CMG-2

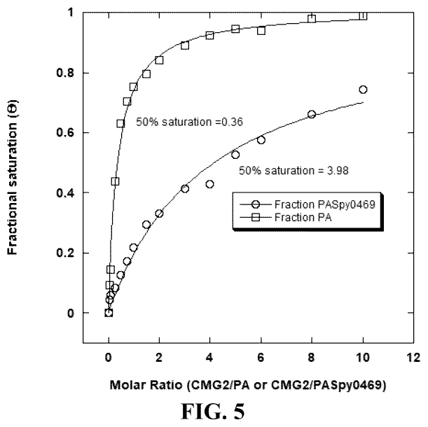

50% saturation of was achieved at a lower molar ratio of CMG-2/PA compared to the molar ratio of CMG-2/PA-Spy0469 (FIG. 5).

### Example 5: Role of CMG-2 in Antigen Presentation

This example presents data in relation to determining antigen presentation of the PA-Spy0469 conjugate to advance anthrax protective antigen (PA) as a platform for development of conjugate vaccines. PA binds with high affinity to cell surface receptor capillary morphogenesis protein 2 (CMG-2). CMG-2 binding of PA is necessary for induction of neutralizing antibodies to anthrax toxin during vaccination of mice with soluble PA antigen independent of adjuvant. To help explain the role of CMG-2 in the generation of neutralizing antibodies, CMG-2 is expressed on human dendritic cells and monocytes, suggesting a role of CMG-2 in PA antigen presentation.

Prior work has shown that antigen conjugated to a non-toxic anthrax lethal factor (LF), the binding partner of heptameric PA, can enter the cytosolic compartment via the PA pore, resulting in activation of both naive CD4+ (MHC-II) and CD8+ (MHC-I) T cells specific for the LF-conjugated antigen. A variation of PA was examined in which conjugated antigen was not expected to enter the cytosol through a heptameric PA pore. Memory T cell frequency was examined in human PBMC samples specific for Spy0469, a S. pyogenes vaccine antigen with high frequency of memory T cell responses in humans because of prior exposure to Group A Streptococcus. A PA conjugate in which Spy0469 is conjugated to PA at the C-terminus of receptor binding domain 4 has been constructed and characterized. The frequency of memory T cells after stimulation with PA, Spy0469, and PA-Spy0469 in human PBMCs of 6 subjects as a source of antigen presenting cells (APCs) were compared. CD4 and CD8 T cells were surface stained for activation markers OX40+/4-1BB+, CD69+/IL-2+, CD69+/IFN-γ+ (CD4+) and CD69+/4-1BB+, CD69+/CD107A+ and CD69+/IFN-γ+ (CD8+) and gated for OX40+/4-1BB+ and CD69+/CD107A+. Data showed the frequency of CD69+/CD107A+ memory CD8 T cells after stimulation with PA, Spy0469, PA-Spy0469, and SEB in human PBMCs of 6 subjects as a source of antigen presenting cells (APCs) and the frequency of OX40+/4-1BB+ memory CD4 T cells after stimulation with PA, Spy0469, PA-Spy0469, and SEB in human PBMCs of 6 subjects as a source of APCs. A distinct enhancement of CD8+ T cells to the conjugated PA-Spy0469 was demonstrated (not shown). This enhancement was particularly observed in 4 of the 6 donors in comparison to samples treated with Spy069 or PA alone.

The anthrax immunization status of any of the 6 donors is not known. In this small sample study, non-responders to Spy0469 or to PA-Spy0469 have not yet been accounted for. Consequently, this is being pursued in a greater number of subjects, anticipating completing a screen of 30 individual donor samples. There is no significant change in stimulated memory CD4+ T cells between Spy0469 (which cannot be targeted to CMG-2 on PBMCs) versus PA-Spy0469 (which can target to CMG-2). Differences in binding affinity of the PA-Spy0469 may account for some differences observed, since the binding affinity to CMG-2 of the PA-Spy0469 conjugate has a ˜10 fold weaker binding affinity in FRET analysis compared to PA alone, likely due to steric effects on the receptor binding domain of PA. It is of interest to extend data that supports enhancement of cross presentation through the class I pathway, which suggests that it may be possible to direct antigens to MHC I and MHCII pathways through endocytic uptake via CMG-2. The data suggest access of antigen to the class I processing pathway by direct entry of the conjugate or a multimer of the conjugate. The mechanism of cross-presentation may be similar to those attributed to other receptors such as CLEC9A, a C-type lectin receptor that has been shown to deliver antigens to both CD4+ and CD8+ pathways.

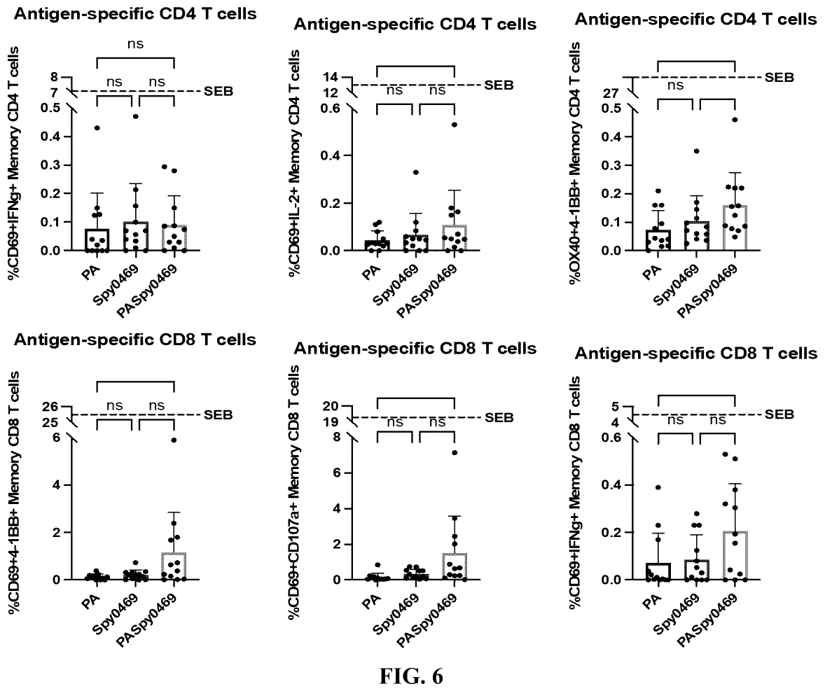

Compiled data for n=12 (fresh human PBMCs) is shown in FIG. 6. PBMCs from 12 healthy individuals were cultured overnight in the presence of 10 μg/ml PA, 5 g/ml Spy0469, or 15 μg/ml PA-Spy0469 conjugate. Cells were then surface stained for markers OX40+/4-1BB+, CD69+/IL-2+, CD69+/IFN-γ+ (CD4+) and CD69+/4-1BB+, CD69+/CD107A+ and CD69+/IFN-γ+ (CD8+). This assay, termed the cytokine-independent activation-induced marker assay (AIM), allows identification of T-cells using markers for specific subsets of T cells using flow cytometry. The background was subtracted (unstimulated) for each donor, and a positive control (Staphylococcus enterotoxin B-SEB, 1 μg/ml) was included in the analysis. *P<0.05, **P<0.01, ns=not significant.

### Example 6: Sequences

-PA Wildtype (reference sequence UniProt: P13423)
SEQ ID NO: 1
EVKQENRLLNESESSSQGLLGYYFSDLNFQAPMVVTSSTTGDLSIPSSELENIPSENQYFQ
SAIWSGFIKVKKSDEYTFATSADNHVTMWVDDQEVINKASNSNKIRLEKGRLYQIKIQY
QRENPTEKGLDFKLYWTDSQNKKEVISSDNLQLPELKQKSSNSRKKRSTSAGPTVPDRD
NDGIPDSLEVEGYTVDVKNKRTFLSPWISNIHEKKGLTKYKSSPEKWSTASDPYSDFEKV
TGRIDKNVSPEARHPLVAAYPIVHVDMENIILSKNEDQSTQNTDSQTRTISKNTSTSRTHT
SEVHGNAEVHASFFDIGGSVSAGFSNSNSSTVAIDHSLSLAGERTWAETMGLNTADTAR
LNANIRYVNTGTAPIYNVLPTTSLVLGKNQTLATIKAKENQLSQILAPNNYYPSKNLAPI
ALNAQDDFSSTPITMNYNQFLELEKTKQLRLDTDQVYGNIATYNFENGRVRVDTGSNW
SEVLPQIQETTARIIFNGKDLNLVERRIAAVNPSDPLETTKPDMTLKEALKIAFGFNEPNG
NLQYQGKDITEFDFNFDQQTSQNIKNQLAELNATNIYTVLDKIKLNAKMNILIRDKRFHY
DRNNIAVGADESVVKEAHREVINSSTEGLLLNIDKDIRKILSGYIVEIEDTEGLKEVINDR
YDMLNISSLRQDGKTFIDFKKYNDKLPLYISNPNYKVNVYAVTKENTIINPSENGDTSTN
GIKKILIFSKKGYEIG
Domain 1, calcium-binding; LF and EF binding sites
SEQ ID NO: 2
EVKQENRLLNESESSSQGLLGYYFSDLNFQAPMVVTSSTTGDLSIPSSELENIPSENQYFQ
SAIWSGFIKVKKSDEYTFATSADNHVTMWVDDQEVINKASNSNKIRLEKGRLYQIKIQY
QRENPTEKGLDFKLYWTDSQNKKEVISSDNLQLPELKQKSSNSRKKRSTSAGPTVPDRD
NDGIPDSLEVEGYTVDVKNKRTFLSPWISNIHEKKGLTKYKSSPEKWSTASDPYSDFEKV
TGRIDKNVSPEARHPLVAA
omain 2, membrane insertion and heptamerization
SEQ ID NO: 3
YPIVHVDMENIILSKNEDQSTQNTDSQTRTISKNTSTSRTHTSEVHGNAEVHASFFDIGGS
VSAGFSNSNSSTVAIDHSLSLAGERTWAETMGLNTADTARLNANIRYVNTGTAPIYNVL
PTTSLVLGKNQTLATIKAKENQLSQILAPNNYYPSKNLAPIALNAQDDFSSTPITMNYNQ
FLELEKTKQLRLDTDQVYGNIATYNFENGRVRVDTGSNWSEVLPQIQET
Domain 3, heptamerization
SEQ ID NO: 4
TARIIFNGKDLNLVERRIAAVNPSDPLETTKPDMTLKEALKIAFGFNEPNGNLQYQGKDI
TEFDFNFDQQTSQNIKNQLAELNATNIYTVLDKIKLNAKMNILIRDKR
Domain 4, binding to the receptor
SEQ ID NO: 5
FHYDRNNIAVGADESVVKEAHREVINSSTEGLLLNIDKDIRKILSGYIVEIEDTEGLKEVIN
DRYDMLNISSLRQDGKTFIDFKKYNDKLPLYISNPNYKVNVYAVTKENTIINPSENGDTS
TNGIKKILIFSKKGYEIG
Uniprot P13423, encoded on Plasmid pXO1 of B. anthracis,
SEQ ID NO: 6
MKKRKVLIPLMALSTILVSSTGNLEVIQAEVKQENRLLNESESSSQGLLGYYFSDLNF
QAPMVVTSSTTGDLSIPSSELENIPSENQYFQSAIWSGFIKVKKSDEYTFATSADNHVTM
WVDDQEVINKASNSNKIRLEKGRLYQIKIQYQRENPTEKGLDFKLYWTDSQNKKEVISS
DNLQLPELKQKSSNSRKKRSTSAGPTVPDRDNDGIPDSLEVEGYTVDVKNKRTFLSPWIS
NIHEKKGLTKYKSSPEKWSTASDPYSDFEKVTGRIDKNVSPEARHPLVAAYPIVHVDME
NIILSKNEDQSTQNTDSQTRTISKNTSTSRTHTSEVHGNAEVHASFFDIGGSVSAGFSNSN
SSTVAIDHSLSLAGERTWAETMGLNTADTARLNANIRYVNTGTAPIYNVLPTTSLVLGK
NQTLATIKAKENQLSQILAPNNYYPSKNLAPIALNAQDDFSSTPITMNYNQFLELEKTKQ
LRLDTDQVYGNIATYNFENGRVRVDTGSNWSEVLPQIQETTARIIFNGKDLNLVERRIAA
VNPSDPLETTKPDMTLKEALKIAFGFNEPNGNLQYQGKDITEFDFNFDQQTSQNIKNQLA
ELNATNIYTVLDKIKLNAKMNILIRDKRFHYDRNNIAVGADESVVKEAHREVINSSTEGL
LLNIDKDIRKILSGYIVEIEDTEGLKEVINDRYDMLNISSLRQDGKTFIDFKKYNDKLPLYI
SNPNYKVNVYAVTKENTIINPSENGDTSTNGIKKILIFSKKGYEIG

The above bolded sequence is the periplasmic signal sequence (SEQ ID NO:7). The remainder of the sequence is PA, wild type.

Coding sequence (GenBank AF306778, Bacillus anthracis plasmid
pX01 protective antigen (pag) gene, complete cds,
SEQ ID NO: 8
aatttcaata taatataaat ttaattttat acaaaaagga gaacgtatat gaaaaaacga
aaagtgttaa taccattaat ggcattgtct acgatattag tttcaagcac aggtaattta
gaggtgattc aggcagaagt taaacaggag aaccggttat taaatgaatc agaatcaagt
tcccaggggt tactaggata ctattttagt gatttgaatt ttcaagcacc catggtggtt
acctcttcta ctacagggga tttatctatt cctagttctg agttagaaaa tattccatcg
gaaaaccaat attttcaatc tgctatttgg tcaggattta tcaaagttaa gaagagtgat
gaatatacat ttgctacttc cgctgataat catgtaacaa tgtgggtaga tgaccaagaa
gtgattaata aagcttctaa ttctaacaaa atcagattag aaaaaggaag attatatcaa
ataaaaattc aatatcaacg agaaaatcct actgaaaaag gattggattt caagttgtac
tggaccgatt ctcaaaataa aaaagaagtg atttctagtg ataacttaca attgccagaa
ttaaaacaaa aatcttcgaa ctcaagaaaa aagcgaagta caagtgctgg acctacggtt
ccagaccgtg acaatgatgg aatccctgat tcattagagg tagaaggata tacggttgat
gtcaaaaata aaagaacttt totttcacca tggatttcta atattcatga aaagaaagga
ttaaccaaat ataaatcatc tcctgaaaaa tggagcacgg cttctgatcc gtacagtgat
ttcgaaaagg ttacaggacg gattgataag aatgtatcac cagaggcaag acaccccctt
gtggcagctt atccgattgt acatgtagat atggagaata ttattctctc aaaaaatgag
gatcaatcca cacagaatac tgatagtcaa acgagaacaa taagtaaaaa tacttctaca
agtaggacac atactagtga agtacatgga aatgcagaag tgcatgcgtc gttctttgat
attggtggga gtgtatctgc aggatttagt aattcgaatt caagtacggt cgcaattgat
cattcactat ctctagcagg ggaaagaact tgggctgaaa caatgggttt aaataccgct
gatacagcaa gattaaatgc caatattaga tatgtaaata ctgggacggc tccaatctac
aacgtgttac caacgacttc gttagtgtta ggaaaaaatc aaacactogc gacaattaaa
gctaaggaaa accaattaag tcaaatactt gcacctaata attattatcc ttctaaaaac
ttggcgccaa togcattaaa tgcacaagac gatttcagtt ctactccaat tacaatgaat
tacaatcaat ttcttgagtt agaaaaaacg aaacaattaa gattagatac ggatcaagta
tatgggaata tagcaacata caattttgaa aatggaagag tgagggtgga tacaggctcg
aactggagtg aagtgttacc gcaaattcaa gaaacaactg cacgtatcat ttttaatgga
aaagatttaa atctggtaga aaggcggata gcggcggtta atcctagtga tccattagaa
acgactaaac cggatatgac attaaaagaa gcccttaaaa tagcatttgg atttaacgaa
ccgaatggaa acttacaata tcaagggaaa gacataaccg aatttgattt taatttcgat
caacaaacat ctcaaaatat caagaatcag ttagcggaat taaacgcaac taacatatat
actgtattag ataaaatcaa attaaatgca aaaatgaata ttttaataag agataaacgt
tttcattatg atagaaataa catagcagtt ggggcggatg agtcagtagt taaggaggct
catagagaag taattaattc gtcaacagag ggattattgt taaatattga taaggatata
agaaaaatat tatcaggtta tattgtagaa attgaagata ctgaagggct taaagaagtt
ataaatgaca gatatgatat gttgaatatt tctagtttac ggcaagatgg aaaaacattt
atagatttta aaaaatataa tgataaatta ccgttatata taagtaatcc caattataag
gtaaatgtat atgctgttac taaagaaaac actattatta atcctagtga gaatggggat
actagtacca acgggatcaa gaaaatttta atcttttcta aaaaaggcta tgagatagga
taaggtaatt ctaggtgatt tttaaatta
Linkers:
(SEQ ID NO: 9)
GSGESGSG,
(SEQ ID NO: 10)
GSGEESGSG,
(SEQ ID NO: 11)
GSGESGSGS,
(SEQ ID NO: 12)
GSGEESGSGS,
and
(SEQ ID NO: 13)
GGGGSGGGGS.
SpyTag:
(SEQ ID NO: 14)
AHIVMVDAYKPT.
SpyCatcher
(SEQ ID NO: 15)
MSYYHHHHHHDYDIPTTENLYFQGAMVDTLSGLSSEQGQSGDMTIEEDSATHIKFSKRD
EDGKELAGATMELRDSSGKTISTWISDGQVKDFYLYPGKYTFVETAAPDGYEVATAITF
TVNEQGQVTVNGKATKGDAHI
Periplasmic-PA
(SEQ ID NO: 16)
MKYLLPTAAA GLLLLAAQPA MAMDIGINSD PM EVKQENRL LNESESSSQG
LLGYYFSDLN FQAPMVVTSS TTGDLSIPSS ELENIPSENQ YFQSAIWSGF IKVKKSDEYT
FATSADNHVT MWVDDQEVIN KASNSNKIRL EKGRLYQIKI QYQRENPTEK
GLDFKLYWTD SQNKKEVISS DNLQLPELKQ KSSNSRKKRS TSAGPTVPDR
DNDGIPDSLE VEGYTVDVKN KRTFLSPWIS NIHEKKGLTK YKSSPEKWST
ASDPYSDFEK VTGRIDKNVS PEARHPLVAA YPIVHVDMEN IILSKNEDQS
TQNTDSQTRT ISKNTSTSRT HTSEVHGNAE VHASFFDIGG SVSAGFSNSN SSTVAIDHSL
SLAGERTWAE TMGLNTADTA RLNANIRYVN TGTAPIYNVL PTTSLVLGKN
QTLATIKAKE NQLSQILAPN NYYPSKNLAP IALNAQDDFS STPITMNYNQ
FLELEKTKQL RLDTDQVYGN IATYNFENGR VRVDTGSNWS EVLPQIQETT
ARIIFNGKDL NLVERRIAAV NPSDPLETTK PDMTLKEALK IAFGFNEPNG
NLQYQGKDIT EFDFNFDQQT SQNIKNQLAE LNATNIYTVL DKIKLNAKMN
ILIRDKRFHY DRNNIAVGAD ESVVKEAHRE VINSSTEGLL LNIDKDIRKI LSGYIVEIED
TEGLKEVIND RYDMLNISSL RQDGKTFIDF KKYNDKLPLY ISNPNYKVNV
YAVTKENTII NPSENGDTST NGIKKILIFS KKGYEIG

The above bolded sequence is the periplasmic secretion sequence (SEQ ID NO:17), and the underlined is the remainder of the in frame sequence (MDIGINSD PM, SEQ ID NO:18) following the secretion signal, remaining as an artifact of the cloning and expression system. The remainder of the sequence is PA, wild type (SEQ ID NO:1).

PA-Spy0469 conjugate
(SEQ ID NO: 19)
MKYLLPTAAA GLLLLAAQPA MAMDIGINSD PMEVKQENRL LNESESSSQG
LLGYYFSDLN FQAPMVVTSS TTGDLSIPSS ELENIPSENQ YFQSAIWSGF IKVKKSDEYT
FATSADNHVT MWVDDQEVIN KASNSNKIRL EKGRLYQIKI QYQRENPTEK
GLDFKLYWTD SQNKKEVISS DNLQLPELKQ KSSNSRKKRS TSAGPTVPDR
DNDGIPDSLE VEGYTVDVKN KRTFLSPWIS NIHEKKGLTK YKSSPEKWST
ASDPYSDFEK VTGRIDKNVS PEARHPLVAA YPIVHVDMEN IILSKNEDQS
TQNTDSQTRT ISKNTSTSRT HTSEVHGNAE VHASFFDIGG SVSAGFSNSN SSTVAIDHSL
SLAGERTWAE TMGLNTADTA RLNANIRYVN TGTAPIYNVL PTTSLVLGKN
QTLATIKAKE NQLSQILAPN NYYPSKNLAP IALNAQDDFS STPITMNYNQ
FLELEKTKQL RLDTDQVYGN IATYNFENGR VRVDTGSNWS EVLPQIQETT
ARIIFNGKDL NLVERRIAAV NPSDPLETTK PDMTLKEALK IAFGFNEPNG
NLQYQGKDIT EFDFNFDQQT SQNIKNQLAE LNATNIYTVL DKIKLNAKMN
ILIRDKRFHY DRNNIAVGAD ESVVKEAHRE VINSSTEGLL LNIDKDIRKI LSGYIVEIED
TEGLKEVIND RYDMLNISSL RQDGKTFIDF KKYNDKLPLY ISNPNYKVNV
YAVTKENTII NPSENGDTST NGIKKILIFS KKGYEIG SSTAQAQEWT PRSVTEIKS
ELVLVDNVFT YTVKYGDTLS TIAEAMGIDV HVLGDINHIA NIDLIFPDTI
LTANYNQHGQ ATNLTVQAPA SSPASVSHVP SSEPLPQASA TSQPTVPMAP
PATPSDVPTT PFASAKPDSS VTASSELTSS TNDVSTELSS ESQKQPEVPQ
EAVPTPKAAE TTEVEPKTDI SEAPTSANRP VPNESASEEV SSAAPAQAPA
EKEETSAPAA QKAVADTTSV ATSNGLSYAP NHAYNPMNAG LQPQTAAFKE
EVASAFGITS FSGYRPGDPG DHGKGLAIDF MVPENSALGD QVAQYAIDHM
AERGISYVIW KQRFYAPFAS IYGPAYTWNP MPDRGSITEN HYDHVHVSEN A

The first bolded sequence is the periplasmic pelA signal sequence, followed by the underlined in frame sequence from cloning. The standard text is PA. The italicized text is the SST linker. The second bolded sequence is Spy0469. Key Spy0469 epitopes are underlined.

His-tagged Spy0469
(SEQ ID NO: 20)
MGHHHHHHGS GENLYFQGSG QAQEWTPRSV TEIKSELVLV DNVFTYTVKY
GDTLSTIAEA MGIDVHVLGD INHIANIDLI FPDTILTANY NQHGQATNLT
VQAPASSPAS VSHVPSSEPL PQASATSQPT VPMAPPATPS DVPTTPFASA
KPDSSVTASS ELTSSTNDVS TELSSESQKQ PEVPQEAVPT PKAAETTEVE
PKTDISEAPT SANRPVPNES ASEEVSSAAP AQAPAEKEET SAPAAQKAVA
DTTSVATSNG LSYAPNHAYN PMNAGLQPQT AAFKEEVASA FGITSFSGYR
PGDPGDHGKG LAIDFMVPEN SALGDQVAQY AIDHMAERGI SYVIWKQRFY
APFASIYGPA YTWNPMPDRG SITENHYDHV HVSFNA

The first bolded sequence is the His-Tag+GSG linker. The standard text is the TEV protease recognition sequence. The italicized text is the GSG linker. The second bolded sequence is Spy0469. Key Spy0469 epitopes are underlined.

Spy0469 (SEQ ID NO: 21), with key Spy0469 epitopes underlined:
QAQEWTPRSV TEIKSELVLV DNVFTYTVKY GDTLSTIAEA MGIDVHVLGD
INHIANIDLI FPDTILTANY NQHGQATNLT VQAPASSPAS VSHVPSSEPL PQASATSQPT
VPMAPPATPS DVPTTPFASA KPDSSVTASS ELTSSTNDVS TELSSESQKQ PEVPQEAVPT
PKAAETTEVE PKTDISEAPT SANRPVPNES ASEEVSSAAP AQAPAEKEET
SAPAAQKAVA DTTSVATSNG LSYAPNHAYN PMNAGLQPQT AAFKEEVASA
FGITSFSGYR PGDPGDHGKG LAIDFMVPEN SALGDQVAQY AIDHMAERGI
SYVIWKQRFY APFASIYGPA YTWNPMPDRG SITENHYDHV HVSFNA
Spy0469 Epitope #1
(SEQ ID NO: 22)
AQEWTPRSVTEIKSELVL,
Spy0469 Epitope #2
(SEQ ID NO: 23)
ATPSDVPTTPFASAKPDSSVTA,
Spy0469 Epitope #3
(SEQ ID NO: 24)
EPKTDISEAPTSANRPVPNESAS.
Spy0469 Epitopes #1-3 are key epitopes of Spy0469.
Spy 1228, sub-sequence of Spy0469
(SEQ ID NO: 25)
EWTPRSV TEIKSELVLV DNVFTYTVKY GDTLSTIAEA MGIDVHVLGD INHIANIDLI
FPDTILTANY NQHGQATNLT VQAPASSPAS VSHVPSSEPL PQASATSQPT
VPMAPPATPS DVPTTPFASA KPDSSVTASS ELTSSTNDVS TELSSESQKQ PEVPQEAVPT
PKAAETTEVE PKTDISEAPT SANRPVPNES ASEEVSSAAP AQAPAEKEET
SAPAAQKAVA DTTSVATSNG LSYAPNHAYN PMNAGLQPQT AAFKEEVASA
FGITSFSGYR PGDPGDHGKG LAIDFMVPEN SALGDQVAQY AIDHMAERGI SYVIWKQ
SpyCatcher003,
SEQ ID NO: 26
SATHIKFSKRDEDGRELAGATMELRDSSGKTISTWISDGHVKDFYLYPGKYTFVETAAP
DGYEVA TPIEFTVNED GQVTV
SpyCatcher003, variant,
SEQ ID NO: 27
MVTTLSGLSGEQGPSGDMTTEEDSATHIKFSKRDEDGRELAGATMELRDSSGKTISTWIS
DGHVKDFYLYPGKYTFVETAAPDGYEVATPIEFTVNEDGQVTVDGEATEGDAHT
PA-SpyCatcher003 conjugate,
SEQ ID NO: 28
MKYLLPTAAA GLLLLAAQPA MAMDIGINSD PMEVKQENRL LNESESSSQG
LLGYYFSDLN FQAPMVVTSS TTGDLSIPSS ELENIPSENQ YFQSAIWSGF
IKVKKSDEYT FATSADNHVT MWVDDQEVIN KASNSNKIRL EKGRLYQIKI
QYQRENPTEK GLDFKLYWTD SQNKKEVISS DNLQLPELKQ KSSNSRKKRS
TSAGPTVPDR DNDGIPDSLE VEGYTVDVKN KRTFLSPWIS NIHEKKGLTK
YKSSPEKWST ASDPYSDFEK VTGRIDKNVS PEARHPLVAA YPIVHVDMEN
IILSKNEDQS TQNTDSQTRT ISKNTSTSRT HTSEVHGNAE VHASFFDIGG
SVSAGFSNSN SSTVAIDHSL SLAGERTWAE TMGLNTADTA RLNANIRYVN
TGTAPIYNVL PTTSLVLGKN QTLATIKAKE NQLSQILAPN NYYPSKNLAP
IALNAQDDFS STPITMNYNQ FLELEKTKQL RLDTDQVYGN IATYNFENGR
VRVDTGSNWS EVLPQIQETT ARIIFNGKDL NLVERRIAAV NPSDPLETTK
PDMTLKEALK IAFGFNEPNG NLQYQGKDIT EFDFNFDQQT SQNIKNQLAE
LNATNIYTVL DKIKLNAKMN ILIRDKRFHY DRNNIAVGAD ESVVKEAHRE
VINSSTEGLL LNIDKDIRKI LSGYIVEIED TEGLKEVIND RYDMLNISSL
RQDGKTFIDF KKYNDKLPLY ISNPNYKVNV YAVTKENTII NPSENGDTST
NGIKKILIFSKKGYEIGGSGESGSGSMVTTLSGLSGEQGPSGDMTTEEDSATHIKFSKRDE
DGRELAGATMELRDSSGKTISTWISDGHVKDFYLYPGKYTFVETAAPDGYEVATPIEFT
VNEDGQVTVDGEATEGDAHT
PA-SpyCatcher003 conjugate,
SEQ ID NO: 29
MKYLLPTAAA GLLLLAAQPA MAMDIGINSD PMEVKQENRL LNESESSSQG
LLGYYFSDLN FQAPMVVTSS TTGDLSIPSS ELENIPSENQ YFQSAIWSGF
IKVKKSDEYT FATSADNHVT MWVDDQEVIN KASNSNKIRL EKGRLYQIKI
QYQRENPTEK GLDFKLYWTD SQNKKEVISS DNLQLPELKQ KSSNSRKKRS
TSAGPTVPDR DNDGIPDSLE VEGYTVDVKN KRTFLSPWIS NIHEKKGLTK
YKSSPEKWST ASDPYSDFEK VTGRIDKNVS PEARHPLVAA YPIVHVDMEN
IILSKNEDQS TQNTDSQTRT ISKNTSTSRT HTSEVHGNAE VHASFFDIGG
SVSAGFSNSN SSTVAIDHSL SLAGERTWAE TMGLNTADTA RLNANIRYVN
TGTAPIYNVL PTTSLVLGKN QTLATIKAKE NQLSQILAPN NYYPSKNLAP
IALNAQDDFS STPITMNYNQ FLELEKTKQL RLDTDQVYGN IATYNFENGR
VRVDTGSNWS EVLPQIQETT ARIIFNGKDL NLVERRIAAV NPSDPLETTK
PDMTLKEALK IAFGFNEPNG NLQYQGKDIT EFDFNFDQQT SQNIKNQLAE
LNATNIYTVL DKIKLNAKMN ILIRDKRFHY DRNNIAVGAD ESVVKEAHRE
VINSSTEGLL LNIDKDIRKI LSGYIVEIED TEGLKEVIND RYDMLNISSL
RQDGKTFIDF KKYNDKLPLY ISNPNYKVNV YAVTKENTII NPSENGDTST
NGIKKILIFS KKGYEIGGSG ESGSGSATHI KFSKRDEDGR ELAGATMELR
DSSGKTISTW ISDGHVKDFY LYPGKYTFVE TAAPDGYEVA TPIEFTVNED
GQVTV

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

1. An immunogenic protein conjugate comprising anthrax toxin component protective antigen (PA), a peptide linker, and an antigen.

2. The immunogenic protein conjugate of claim 1, wherein the antigen comprises an antigen from a group A Streptococcus species, Streptococcus pneumoniae, Mycobacterium tuberculosis, a malaria-causing Plasmodium species, or Human immunodeficiency virus (HIV), human cytomegalovirus, Borrelia burgdorferi, Newcastle Disease Virus, or a cancer.

3. The immunogenic protein conjugate of claim 1, wherein the antigen comprises an antigen from Streptococcus pyogenes.

4. The immunogenic protein conjugate of claim 1, wherein the antigen comprises Spy0469, Spy1228, Spy003, ESAT-6 (Early Secreted Antigenic Target 6), Ag85B (Mycolyl transferase), HtpG (Heat shock protein), gp120 (Envelope glycoprotein), Gag (Group-specific antigen), Retanef (Fusion of Rev, Tat, Nef), SpyCEP (Streptococcal protease), C5a Peptidase (ScpA), SpyAD (Adhesin), HER2/neu (Human Epidermal Growth Factor Receptor 2), MUC1 (Mucin 1), MAGE-A3 (Melanoma-Associated Antigen A3), NY-ESO-1 (New York Esophageal Squamous Cell Carcinoma-1), WT1 (Wilms' Tumor 1), or p53 (Tumor Protein 53).

5. The immunogenic protein conjugate of claim 1, wherein the linker comprises a peptide of from 2 to 10 amino acid residues, or a system comprising a specific binding pair, such as a SpyTag peptide and a SpyCatcher peptide.

6. The immunogenic protein conjugate of claim 1, wherein the PA comprises an amino acid sequence of SEQ ID NO: 1, or a sequence have 95% identity to SEQ ID NO:1.

7. The immunogenic protein conjugate of claim 1, wherein the protein conjugate further comprises a periplasmic signal sequence.

8. The immunogenic protein conjugate of claim 1, wherein the protein conjugate comprises SEQ ID NO: 19, 28, or 29.

9. The immunogenic protein conjugate of claim 1, wherein the linker is attached to domain 4 of PA.

10. A plasmid encoding the immunogenic protein conjugate of claim 1.

11. An immunogenic composition comprising a therapeutically effective amount of the immunogenic protein conjugate of claim 1 dispersed in a pharmaceutically acceptable carrier.

12. The immunogenic composition of claim 11, wherein said composition is a group A Streptococcus vaccine.

13. The immunogenic composition of claim 11, wherein said composition is a Streptococcus pyogenes vaccine.

14. A method of inducing an immune response against an antigenic agent, the method comprising administering a therapeutically effective amount of an immunogenic protein conjugate to a subject in need thereof, wherein said immunogenic protein conjugate comprises anthrax toxin component protective antigen (PA), a peptide linker, and an antigen, wherein said subject has an increased immune response against said antigen as compared to an unvaccinated control.

15. The method of claim 14, wherein said increased immune response comprises activation of CD4+ and CD8+ T cells in said subject.

16. The method of claim 14, wherein said increased immune response comprises induction of serum antibodies against said antigen in said subject.

17. The method of claim 14, wherein said antigen is derived from an infectious or pathogenic virus or microorganism.

18. The method of claim 17, wherein said immunogenic protein conjugate is administered before said subject is exposed to or at risk of exposure to said infectious or pathogenic virus or microorganism.

19. The method of claim 17, wherein said immunogenic protein conjugate is administered to said subject is a priming dose or as a booster vaccine against said infectious or pathogenic virus or microorganism.

20. The method of claim 17, wherein said immunogenic protein conjugate is administered via injection.
